(19) United States
(12) Patent Application Publication
(10) Pub. No.: US 2025/0204870 A1
(43) Pub. Date: Jun. 26, 2025

(54) X-RAY IMAGING APPARATUS

(71) Applicants: RAYENCE Co., Ltd., Gyeonggi-do (KR); VATECH EWOO Holdings Co., Ltd., Gyeonggi-do (KR)

(72) Inventors: Tae Woo KIM, Gyeonggi-do (KR); Soo Hwa KAM, Gyeonggi-do (KR)

(73) Assignees: RAYENCE Co., Ltd., Gyeonggi-do (KR); VATECH EWOO Holdings Co., Ltd., Gyeonggi-do (KR)

(21) Appl. No.: 18/990,660

(22) Filed: Dec. 20, 2024

(30) Foreign Application Priority Data
Dec. 22, 2023 (KR) 10-2023-0190524

(51) Int. Cl. A61B 6/02; A61B 6/00

(52) U.S. Cl. CPC A61B 6/025; A61B 6/027; A61B 6/4441; A61B 6/4452; A61B 6/5205; A61B 6/54

(57) ABSTRACT

Proposed is a C-arm X-ray imaging apparatus that allows dual-energy tomosynthesis (Tomo) imaging using the same driving system as regular Tomo imaging. The apparatus includes a C-shaped arm, an X-ray generator disposed at a first end of the C-shaped arm, and an X-ray detector disposed at a second end of the C-shaped arm to face the X-ray generator with a subject in between, wherein the X-ray generator moves back and forth between first and second positions on opposite sides of an initial position and selectively emits X-rays of different first and second energy levels toward the subject, and the X-ray detector receives the X-rays of the first and second energy levels that have passed through the subject.

### CROSS REFERENCE TO RELATED APPLICATION

The present application claims priority to Korean Patent Application No. 10-2023-0190524, filed Dec. 22, 2023, the entire contents of which is incorporated herein for all purposes by this reference.

### BACKGROUND

### Technical Field

The present disclosure relates to an X-ray imaging apparatus in which an X-ray generator and an X-ray detector are respectively disposed at opposite ends of a C-shaped arm.

### Description of the Related Art

An X-ray machine is a device that emits X-rays toward a subject and obtains an X-ray image from the X-rays that travel through the subject to display the internal structure of the subject.

An example of the X-ray machine is an X-ray fluoroscopy apparatus, commonly called a C-arm, which is used in operating rooms, emergency rooms, or during other clinical procedures. As an example, the C-arm X-ray machine consists of a mobile base, a multi-joint arm supporter connected to the mobile base, and a C-shaped arm connected to the multi-joint arm supporter. Additionally, an X-ray generator and an X-ray detector are respectively installed at opposite ends of the C-shaped arm to capture an X-ray fluoroscopy image of an object placed therebetween.

The C-arm X-ray machine is mainly used to acquire two-dimensional X-ray fluoroscopy images of a subject. Meanwhile, in order to check the exact location of a lesion, surgical progress, location of a surgical instrument, and degree of fastening during surgery, tomographic images may be required. However, it is difficult to move a patient to an imaging room equipped with a CT imaging system during surgery to obtain tomographic images. A C-arm X-ray machine with a tomosynthesis function, which involves acquiring multiple tomographic images of an object by emitting X-rays from different angles within a limited angular range, can be an alternative.

The problem is that, to obtain a tomographic image from tomosynthesis, the X-ray generator must rotate and move to obtain multiple projection images of a subject over a certain angle range. In a typical C-arm X-ray machine, because an X-ray generator is fixed to one end of a C-shaped arm, the entire C-shaped arm needs to be rotated to obtain multiple projection images in a certain angle range. In the case of a conventional C-arm X-ray machine with a large and complex driving mechanism, because an entire C-shaped arm needs to be rotated and moved, the imaging time is long, precise control is difficult, and due to increased load and volume, it is often difficult to use the machine in an operating room or even get the machine into the operating room.

### Document of Related Art

(Patent Document 1) Korean Patent Application Publication No. 10-2013-0058633

(Patent Document 2) U.S. Patent Application Publication No. 2020-0085390

(Patent Document 3) Japanese Patent Application Publication No. 2001-133554

(Patent Document 4) Korean Patent Application Publication No. 10-2016-0056194

### SUMMARY

Accordingly, the present disclosure has been made keeping in mind the above problems occurring in the related art, and the present disclosure is intended to provide a C-arm X-ray imaging apparatus that allows tomosynthesis imaging even in a tight space such as an operating room and a treatment/procedure room, that prevents the movement of a driving system from being exposed externally during tomosynthesis imaging, and that allows tomosynthesis imaging at different energy levels with just one cycle.

In order to achieve the above objective, according to an aspect of the present disclosure, there is provided an X-ray imaging apparatus including: a C-shaped arm; an X-ray generator disposed at a first end of the C-shaped arm; and an X-ray detector disposed at a second end of the C-shaped arm to face the X-ray generator with a subject in between, wherein the X-ray generator may move back and forth between first and second positions on opposite sides of an initial position and may selectively emit X-rays of different first and second energy levels toward the subject, and the X-ray detector may receive the X-rays of the first and second energy levels that have passed through the subject.

The X-ray generator may travel back and forth through a first section between the initial position and the first position and a second section between the initial position and the second position once each, and may emit X-rays of any one of the first and second energy levels when moving through each of the first and second sections in a first direction and emit X-rays of remaining one of the first and second energy levels when moving in a second direction.

At this time, when the X-ray generator travels back and forth through the first section starting from the initial position, passes the initial position, and proceeds to the second section, the X-ray generator may switch an energy level of the X-rays from one of the first and second energy levels to the remaining one based on the initial position.

In addition, when the X-ray generator travels back and forth through the first section starting from the initial position, passes the initial position, and proceeds to the second section, the X-ray generator may keep an energy level of the X-rays same as one of the first and second energy levels before and after passing the initial position.

The X-ray imaging apparatus may further include a controller configured to reconstruct multiple tomographic images of the subject based on detection results of the X-ray detector, wherein the controller may reconstruct multiple tomographic images of the first and second energy levels for the subject based on the results of detecting X-rays of the first and second energy levels.

In addition, the X-ray imaging apparatus may further include a guide rail connected to the first end of the C-shaped arm, wherein the X-ray generator may move along the guide rail.

In this case, the guide rail may be arched, and the X-ray generator may move along the guide rail while maintaining an equidistant distance from the X-ray detector.

In order to achieve the above objective, according to an aspect of the present disclosure, there is provided an X-ray imaging method using an X-ray imaging apparatus that includes: a C-shaped arm; an X-ray generator disposed at a first end of the C-shaped arm, and configured to move back and forth between first and second positions on opposite sides of an initial position and to selectively emit X-rays of different first and second energy levels; and an X-ray detector disposed at a second end of the C-shaped arm to face the X-ray generator with a subject in between, and configured to receive the X-rays of the first and second energy levels that have passed through the subject, wherein the X-ray generator may travel back and forth through a first section from the initial position to the first position and a second section from the initial position to the second position at least once, and may emit the X-rays of the first and second energy levels for each section at least once.

The X-ray generator may emit X-rays of the first energy level while moving along a first direction in each of the first and second sections, and may emit X-rays of the second energy level different from the first energy level while moving along a second direction.

According to the present disclosure, provided is a C-arm X-ray imaging apparatus that allows tomosynthesis imaging even in a tight space such as an operating room and a treatment/procedure room, that does not expose the movement of a driving system during tomosynthesis, and that allows tomosynthesis imaging at different energy levels with just one cycle.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 shows a state in which a C-arm of an X-ray imaging apparatus according to an embodiment of the present disclosure is unfolded forward;

FIG. 2 shows the principle of tomosynthesis imaging of the X-ray imaging apparatus embodiment of the present disclosure;

FIG. 3 shows a state in which a generator casing is removed from the X-ray imaging apparatus according to an embodiment of the present disclosure;

FIG. 4 shows a driving system of a generator part in the embodiment of FIG. 3;

FIG. 5 schematically shows a tomosynthesis imaging sequence of an X-ray imaging apparatus according to an embodiment of the present disclosure;

FIG. 6 schematically shows an example of a tomosynthesis imaging sequence of an X-ray imaging apparatus according to an embodiment of the present disclosure;

FIG. 7 schematically shows another example of a tomosynthesis imaging sequence of an X-ray imaging apparatus according to an embodiment of the present disclosure; and

FIG. 8 shows the tomosynthesis image acquisition order of the X-ray imaging apparatus according to an embodiment of the present disclosure.

### DETAILED DESCRIPTION

Hereinafter, embodiments of the present disclosure will be described in detail with reference to the drawings. The technical idea of the present disclosure may be more clearly understood through the embodiments. The present disclosure is not limited to the embodiments described below.

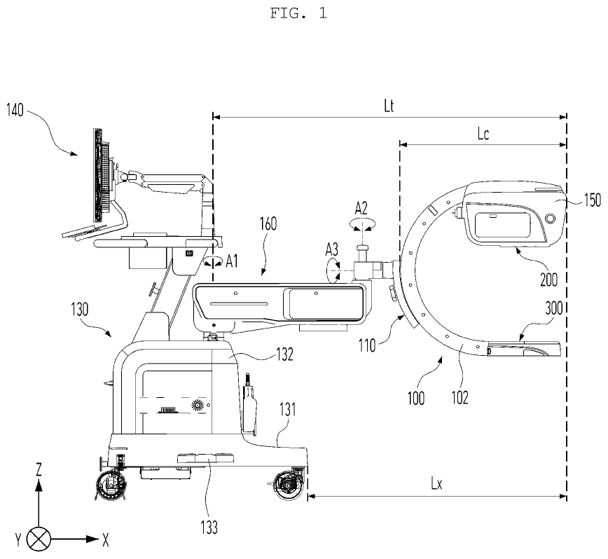

FIG. 1 shows a state in which a C-arm of an X-ray imaging apparatus according to an embodiment of the present disclosure is unfolded forward.

In FIG. 1 and the following drawings, the x-axis direction represents the front of the X-ray imaging apparatus, the y-axis direction represents a direction perpendicular to the x-axis on the plane where the X-ray imaging apparatus is placed, and the z-axis direction represents a direction perpendicular to the plane.

The X-ray imaging apparatus according to this embodiment of the present disclosure largely include a main body part 130, a C-arm part 100, and a joint arm 160 connecting the main body part 130 and the C-arm part 100. A mobile base 131 is provided at the lower part of the main body part 130 to support the entire load of the apparatus and to be movable. Additionally, the main body part 130 may have a controller 140, such as a console PC, responsible for apparatus control and image processing. The controller 140 may include a display for outputting an image, and an input part where user manipulation is input.

The mobile base 131 may include: wheels respectively arranged at the four corners thereof for stable movement; and a frame supporting the wheels.

The rear end of the joint arm 160 is connected to the main body part 130 and the front end of the joint arm 160 is connected to the C-arm part 100. To be specific, the rear end of the joint arm 160 is connected to the main body part 130 so as to rotate up, down, left, and right within at least a predetermined angle range about a first axis Al parallel to the z-axis. The front end of the joint arm 160 and the C-arm part 100 are connected through a C-arm supporter 110.

The C-arm part 100 includes an X-ray generator 200 (hereinafter referred to as generator), an X-ray detector 300 (hereinafter referred to as detector), and a C-shaped arm 102 with the X-ray generator 200 and the X-ray detector 300 respectively disposed at opposite ends.

The C-arm supporter 110 has one side thereof supporting C-shaped curved surface of the C-shaped arm 102 so that the C-shaped curved surface may slide, and has the other side thereof connected to the front end of the joint arm 160 through a second axis A2 to enable left and right rotation with respect to the front end of the arm part 160. One side of the C-arm supporter 110 connected to the C-shaped arm 102 may be rotatably connected to the other side of the C-arm supporter 110 connected to the joint arm 160 about a third axis A3 perpendicular to the second axis A2.

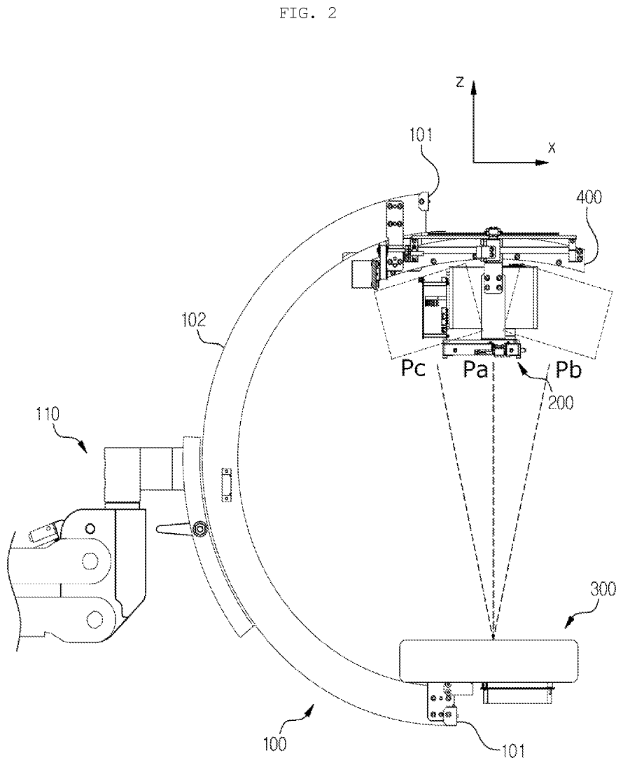

FIG. 2 shows the principle of tomosynthesis imaging of the X-ray imaging apparatus according to an embodiment of the present disclosure. FIG. 2 shows the range of movement of the generator 200 during tomosynthesis imaging with a generator casing 150 removed.

The C-shaped arm 102 of the C-arm part 100 may slide along a C-shaped curve with respect to the C-arm supporter 110, and a stopper 101 is provided at each end of the C-shaped arm 102 to limit the range of sliding.

In the C-arm part 100, an arched guide rail 400 is disposed at one end of the C-shaped arm 102 in the front-rear direction of the extension thereof, and the generator 200 is arranged to move forward and backward along the arched guide rail 400. At this time, the curvature of the arched guide rail 400 is determined so that the generator 200 may move while maintaining an equal distance from the detector 300. In FIG. 2, the outline of the generator 200 indicated by a solid line represents an initial position Pa at the start of tomosynthesis imaging, the outline of the generator 200 indicated by a thin dotted line represents positions Pb and Pc at both ends of the maximum range over which the generator 200 may move, and the thick dotted line represents the change in the irradiation angle of an X-ray beam emitted when the generator 200 is located at the initial position Pa and the positions Pb and Pc at both front and rear ends on the arched guide rail 400.

When the generator 200 moves along the guide rail 400 and emits X-rays at various angles to a subject (not shown) placed on the detector 300, the detector 300 may receive the X-rays at various angles that have passed through the subject, and the controller may reconstruct a plurality of tomographic images of the subject using the light reception results of the detector 300, that is, projection data detected at various angles on the subject.

Meanwhile, the initial position Pa may be the center of the arched guide rail 400, and for tomosynthesis imaging, the generator 200 may be configured to move from the initial position Pa to the front end Pb or rear end Pc and then move to the rear end Pc or front end Pb on the opposite side. The front end Pb and the rear end Pc do not necessarily have to be the ends of the arched guide rail 400, and should be understood to mean opposite ends of the range in which the generator 200 moves. The movement order of the generator 200 during tomosynthesis filming will be discussed in more detail later.

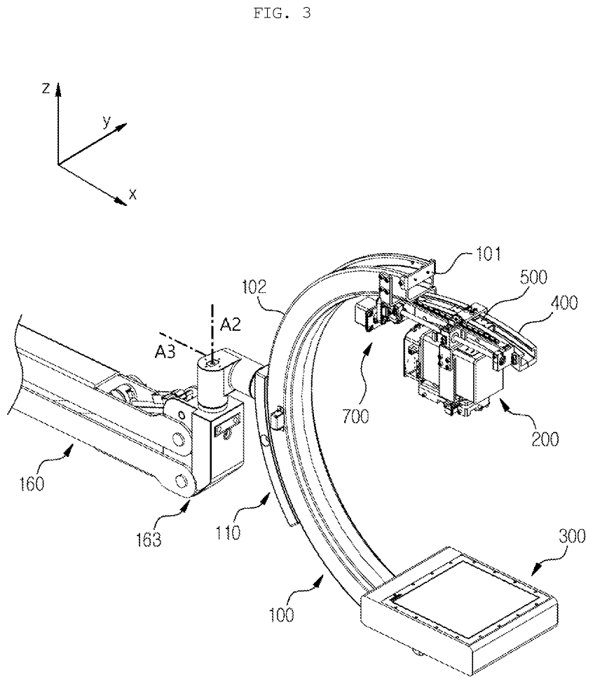

FIG. 3 shows a state in which a generator casing is removed from the X-ray imaging apparatus according to an embodiment of the present disclosure.

Referring to FIG. 3, the driving mechanism of the generator 200 may be explained in more detail. The X-ray imaging apparatus according to an embodiment of the present disclosure includes a driving part 700 that provides power to move the generator 200. The driving part 700 may include a motor. In addition, the driving part 700 may be configured to convert the rotational motion of the motor into linear motion, and transmit the linear motion back to a moving block 500 disposed within the arched guide rail 400 to convert the transmitted linear motion into arc-shaped trajectory motion. The generator 200 is fixed to the moving block 500 and moves together with the moving block 500 along the arched guide rail 400. However, the power transmission method is not limited and may be implemented in various ways, such as directly transmitting the power of the driving motor to the arc-shaped trajectory motion using a flexible power transmission means such as a chain or timing belt.

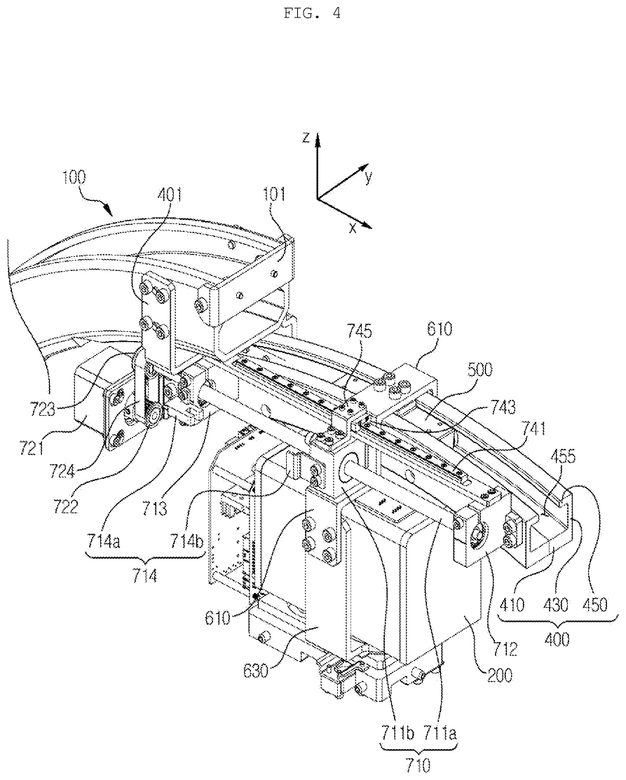

FIG. 4 shows a driving system of a generator part in the embodiment of FIG. 3.

Referring to FIG. 4 together with FIG. 3, the driving part 700 may include a conversion converts rotational motion (lead screw, cam, pinion, etc.) into linear motion (nut, piston, rack, etc.). According to the embodiment, the conversion part 710 may be composed of a combination of a lead screw 711a and a nut 711b. The conversion part 710 may include first and second bearing parts 712 and 713 respectively installed at the front and rear of one side of the arched guide rail 400. The conversion part 710 may include the lead screw 711a. The lead screw 711a may have male threads formed on the outer peripheral surface of the shaft thereof. The lead screw 711a may be rotatably supported on the first and second bearing parts 712 and 713. The conversion part 710 may include the nut 711b. The nut 711b may be fastened to the lead screw 711a. The nut 711b may be a slider that moves linearly back and forth according to the rotation direction of the lead screw 711a.

The nut 711b may include a limit switch. The limit switch may be a switch that opens and closes an electrical contact when a portion of a mechanical part or an object reaches a predetermined position. The limit switch may be composed of a mechanical limit switch, a beam-type limit switch, or a proximity switch. The limit switch of the embodiment may be configured as a proximity switch. The proximity switch may operate when an object approaches a certain distance using magnetism, capacitance, or electromagnetic induction. The proximity switch 714 may include a first proximity switch 714a. The first proximity switch 714a may be installed in the first and second bearing parts 712 and 713. The proximity switch 714 may include a second proximity switch 714b. The second proximity switch 714b may be installed on the nut 711b. The second proximity switch 714b may be installed on the side of the nut 711b. When the first proximity switch 714a detects the second proximity switch 714b, the nut 711b may stop.

The driving part 700 may include a power transmission part that transmits rotational power to the conversion part 710. The power transmission part may include a motor 721 that provides rotational power to the lead screw 711a. The motor 721 may be supported on the C-shaped arm 100 or the arched guide rail 400. The power transmission part may include a driving pulley 722. The driving pulley 722 may be connected to the rotation shaft of the motor 721. The power transmission part 720 may include a driven pulley 723. The driven pulley 723 may be connected to the rear of the lead screw 711a. The power transmission part 720 may include a belt 724. The belt 724 may span the driving pulley 722 and the driven pulley 723. If the driving pulley 722 and the driven pulley 723 are toothed pulleys, the belt 724 may be configured as a timing belt. The nut 711b may reciprocate linearly along the lead screw 711a according to the rotation direction of the motor 721.

The driving part 700 may include a lifting guide part. The lifting guide part may guide a moving bracket 600 up and down in the vertical direction according to the linear reciprocating movement of the conversion part 710.

Due to the configuration illustrated above, the X-ray imaging apparatus according to an embodiment of the present disclosure may perform tomosynthesis imaging by moving the generator 200 between the front end Pb and the rear end Pc of the arched guide rail 400 including the initial position Pa.

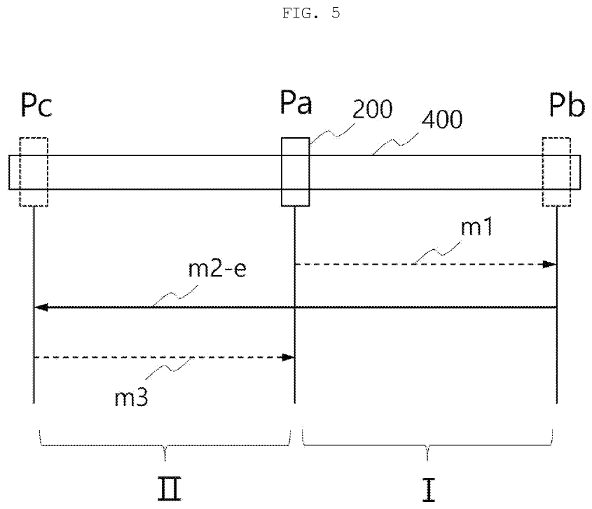

FIG. 5 schematically shows a tomosynthesis imaging sequence of an X-ray imaging apparatus according to an embodiment of the present disclosure.

As previously described, the generator 200 may move along the arched guide rail 400 between the front end Pb and the rear end Pc of the arched guide rail 400. The initial position Pa of the generator 200 may be any point between the front end Pb and the rear end Pc. The initial position Pa may be the center of the arched guide rail 400to quickly respond to various types of imaging such as fluoroscopy, but is not limited thereto.

In the case of general tomosynthesis imaging using single-energy X-rays, the generator 200 first moves (m1) through a first section I between the initial position Pa and the front end Pb without emitting X-rays. Then, the generator 200 moves (m2-e) from the front end Pb to the rear end Pc, that is, through the first section I and a second section II between the initial position Pa and the rear end Pc while periodically or continuously emitting X-rays with a predetermined energy level e. At this time, the detector 300 (see FIG. 2) receives X-rays emitted from various angles depending on the position of the generator 200 and acquires multiple frames of X-ray projection images. After this process is completed, the generator 200 again moves (m3) through the second section II without emitting X-rays to return to the initial position Pa.

In FIG. 5, the m1 and m3 indicated by dotted arrows represent the movement sections through which the generator 200 moved without emitting X-rays, and the m2-e indicated by a solid arrow represents the movement section in which the generator moved while emitting X-rays of a predetermined energy level e. Through this process, multiple tomographic images of the subject may be acquired using multiple frames of X-ray projection images taken in the first section I and the second section II.

In the embodiment, although the generator 200 first moved from the initial position Pa to the front end Pb and then moved toward the rear end Pc, the order may be changed so that the generator 200 moves to the rear end Pc and then moves toward the front end Pb.

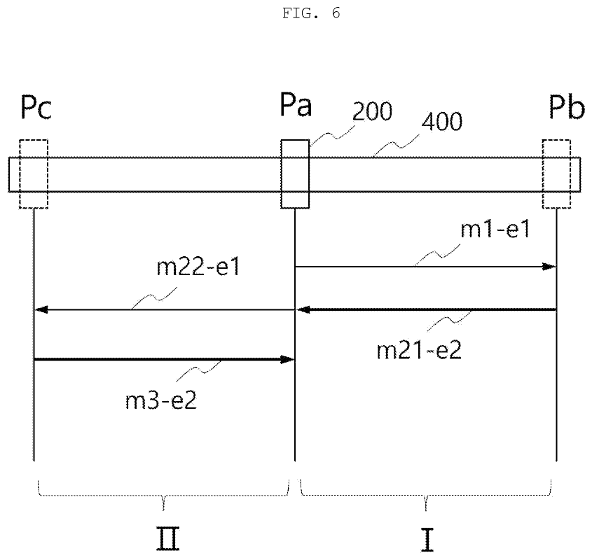

FIG. 6 schematically shows an example of a tomosynthesis imaging sequence of an X-ray imaging apparatus according to an embodiment of the present disclosure.

Dual-energy X-ray imaging refers to a technique of acquiring images at different energy levels of the same subject using X-ray beams with different energy levels. In X-ray images with a relatively high energy level, hard tissues such as bones are emphasized, and in X-ray images with a relatively low energy level, images with soft tissues such as organs and muscles are emphasized. By using X-ray images of different energy levels for the same subject, separate images of hard and soft tissues can be obtained, allowing a targeted lesion, etc. to be identified more accurately.

According to an embodiment of the present disclosure, dual-energy tomosynthesis imaging may be performed in one shot using X-rays of a first energy level e1 and X-rays of a second energy level e2, which have different energy levels. The first and second energy levels e1 and e2 may be obtained by adjusting the voltage and/or current applied to the generator or changing a filter.

First, with the generator 200 located at the initial position Pa on the arched guide rail 400, the generator 200 moves (m1-e1) through the first section I between the initial position Pa and the front end Pb while emitting X-rays of the first energy level e1. The generator 200 may emit X-rays periodically or continuously, and the detector 300 (see FIG. 2) may receive X-rays emitted from various angles depending on the position of the generator 200 in conjunction with X-ray irradiation, and acquire multiple frames of X-ray projection images of the first energy level el for the first section I. The X-ray irradiation method and the method of acquiring X-ray projection images of multiple frames linked thereto are the same in the process described below.

Thereafter, the generator 200 moves (m21-e2) in the opposite direction through the first section I from the front end Pb to the initial position Pa while emitting X-rays of the second energy level e2. In this process, the detector 300 acquires multiple frames of X-ray projection images of the second energy level e2 for the first section I.

Next, the generator 200 moves (m22-e1) through the second section II from the initial position Pa to the rear end Pc while emitting X-rays of the first energy level e1, and the detector 300 acquires multiple frames of X-ray projection images of the first energy level el for the second section II from various angles.

Finally, the generator 200 moves (m3-e2) again through the second section II from the rear end Pc to the initial position Pa of the arched guide rail 400 while emitting X-rays of the second energy level e2, and the detector 300 acquires multiple frames of X-ray projection images of the second energy level e2 for the second section II from various angles. For reference, one of the first and second energy levels represents a relatively high energy level, and the other represents a relatively low energy level.

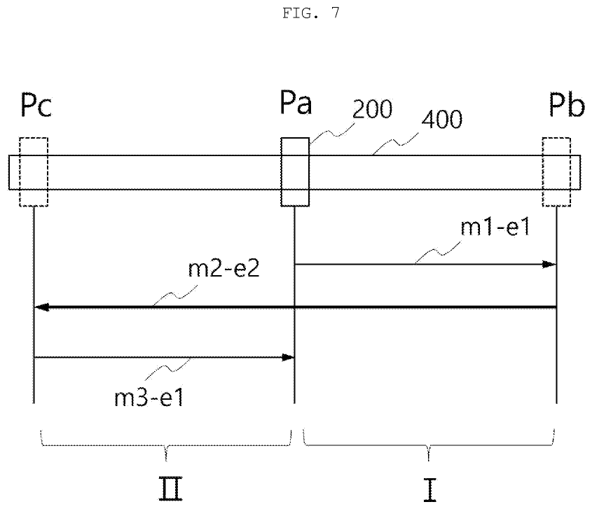

FIG. 7 schematically shows another example of a tomosynthesis imaging sequence of an X-ray imaging apparatus according to an embodiment of the present disclosure.

In the X-ray imaging apparatus according to the embodiment, the generator 200 moves (m1-e1) through the first section I between the initial position Pa and the front end Pb while emitting X-rays of the first energy level e1. In this process, the detector 300 acquires multiple frames of X-ray projection images of the first energy level e1 for the first section I.

Then, the generator 200 moves (m2-e2) continuously in the opposite direction through the first section I and the second section II from the front end Pb to the rear end Pc while emitting X-rays of the second energy level e2. In this process, the detector 300 acquires multiple frames of X-ray projection images of the second energy level e2 for the first section I and the second section II.

Finally, the generator 200 moves (m3-e1) again through the second section II from the rear end Pc to the initial position Pa while emitting X-rays of the first energy level e1, and the detector 300 acquires multiple frames of X-ray projection images of the first energy level e1 for the second section II from various angles.

For reference, one of the first and second energy levels represents a relatively high energy level, and the other represents a relatively low energy level.

As seen with reference to FIGS. 6 and 7, the X-ray imaging apparatus according to an embodiment of the present disclosure may acquire multiple X-ray projection images of the first energy level e1 and the second energy level e2 at different angles for the entire first section I and the second section II through one shooting sequence, and with the multiple X-ray projection images, multiple tomographic images of the first energy level e1 and the second energy level e2 may be acquired for the same subject.

In the above-described embodiments, although the generator 200 first moved from the initial position Pa to the front end Pb, then to the rear end Pc, and then moved back to the initial position Pa, the order may be changed so that the generator 200 moves to the rear end Pc, then moves toward the front end Pb, and finally return to the initial position Pa.

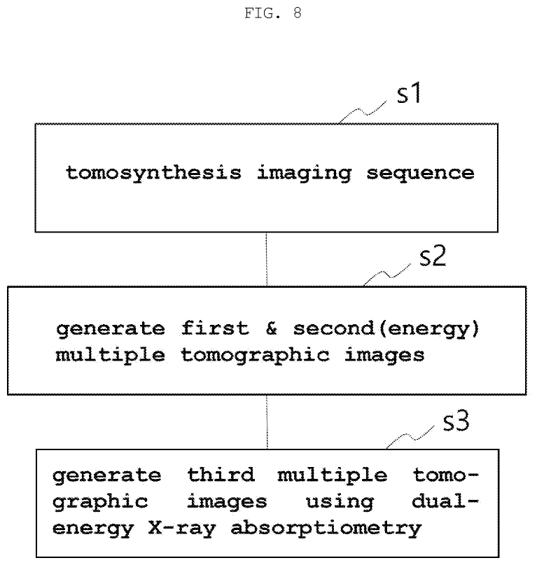

FIG. 8 shows the tomosynthesis image acquisition order of the X-ray imaging apparatus according to an embodiment of the present disclosure.

First, through the dual-energy tomosynthesis imaging sequence according to the embodiment of FIG. 6 or FIG. 7 described above, X-ray projection image groups of the first energy level e1 and the second energy level e2 may be acquired for each of the entire first section I and the second section II (s1).

Then, multiple tomographic images of the first energy level for the subject may be reconstructed using the X-ray projection image groups of the first energy level e1, and multiple tomographic images of the second energy level for the subject may be reconstructed using the X-ray projection image groups of the second energy level e2 (s2).

Thereafter, if necessary, by using dual-energy X-ray absorptiometry, etc., clear images with separated hard or soft tissues may be obtained depending on the purpose with multiple tomographic images of the first energy level and multiple tomographic images of the second energy level. For reference, the dual-energy X-ray absorptiometry may be a method of assigning appropriate weights to X-ray images of different energy levels for subtraction.

Meanwhile, the above description uses the case where a generator is separate from a C-shaped arm and takes tomosynthesis images while moving along a guide rail connected to the end of the C-shaped arm as an example, but can be equally applied to the case where the generator is fixed to the end of the end of the C-shaped arm and moves along with the C-shaped arm, that is, the entire C-arm part to perform tomosynthesis imaging.

What is claimed is:

1. An X-ray imaging apparatus comprising:
a C-shaped arm;
an X-ray generator disposed at a first end of the C-shaped arm; and
an X-ray detector disposed at a second end of the C-shaped arm to face the X-ray generator with a subject in between,
wherein the X-ray generator moves back and forth between first and second positions on opposite sides of an initial position and selectively emits X-rays of different first and second energy levels toward the subject, and
the X-ray detector receives the X-rays of the first and second energy levels that have passed through the subject.

2. The apparatus of claim 1, wherein the X-ray generator travels back and forth through a first section between the initial position and the first position and a second section between the initial position and the second position once each, and emits X-rays of any one of the first and second energy levels when moving through each of the first and second sections in a first direction and emits X-rays of remaining one of the first and second energy levels when moving in a second direction.

3. The apparatus of claim 2, wherein when the X-ray generator travels back and forth through the first section starting from the initial position, passes the initial position, and proceeds to the second section, the X-ray generator switches an energy level of the X-rays from one of the first and second energy levels to the remaining one based on the initial position.

4. The apparatus of claim 2, wherein when the X-ray generator travels back and forth through the first section starting from the initial position, passes the initial position, and proceeds to the second section, the X-ray generator keeps an energy level of the X-rays same as one of the first and second energy levels before and after passing the initial position.

5. The apparatus of claim 1, further comprising:
a controller configured to reconstruct multiple tomographic images of the subject based on detection results of the X-ray detector,
wherein the controller reconstructs multiple tomographic images of the first and second energy levels for the subject based on the results of detecting X-rays of the first and second energy levels.

6. The apparatus of claim 1, further comprising:
a guide rail connected to the first end of the C-shaped arm,
wherein the X-ray generator moves along the guide rail.

7. The apparatus of claim 6, wherein the guide rail is arched, and the X-ray generator moves along the guide rail while maintaining an equidistant distance from the X-ray detector.

8. An X-ray imaging method using an X-ray imaging apparatus that includes: a C-shaped arm; an X-ray generator disposed at a first end of the C-shaped arm, and configured to move back and forth between first and second positions on opposite sides of an initial position and to selectively emit X-rays of different first and second energy levels; and an X-ray detector disposed at a second end of the C-shaped arm to face the X-ray generator with a subject in between, and configured to receive the X-rays of the first and second energy levels that have passed through the subject, wherein the X-ray generator travels back and forth through a first section from the initial position to the first position and a second section from the initial position to the second position at least once, and emits the X-rays of the first and second energy levels for each section at least once.

9. The method of claim 8, wherein the X-ray generator emits X-rays of the first energy level while moving along a first direction in each of the first and second sections, and emits X-rays of the second energy level different from the first energy level while moving along a second direction.
